(19) United States
(12) Patent Application Publication
(10) Pub. No.: US 2017/0355655 A1
(43) Pub. Date: Dec. 14, 2017

(54) PROCESS FOR LIQUID-LIQUID EXTRACTION OF A BLEND OF NON-UNIFORM OLIGOMERS AND POLYMERS

(71) Applicant: SHELL OIL COMPANY, HOUSTON, TX (US)

(72) Inventors: Jean Paul Andre Marie Joseph Ghislain LANGE, Amsterdam (NL); Guus VAN ROSSUM, Amsterdam (NL); Sascha Reinier Aldegonda KERSTEN, Enschede (NL); Shushil KUMAR, Enschede (NL)

(21) Appl. No.: 15/540,821

(22) PCT Filed: Dec. 24, 2015
(86) PCT No.: PCT/EP2015/081240

§ 371 Date: Jun. 29, 2017

(30) Foreign Application Priority Data
Dec. 30, 2014 (EP) 14200663.4

(51) Int. Cl. C07C 7/10; C10G 1/00; C10G 1/06; B01D 11/04

(52) U.S. Cl. CPC C07C 7/10; B01D 11/0492; C10G 1/002; C10G 1/065; C10G 2300/44; C10G 2300/1011

(57) ABSTRACT

A process for liquid-liquid extraction of an oil-blend of non-uniform oligomeric and polymeric components comprising: (a) preselecting a desired molecular weight (Mw) boundary between heavy and light components; (b) selecting an extractive solvent or an extractive mixture of solvents, which form essentially a single phase with the light components; (c) mixing the oil-blend and the extractive solvent or extractive mixture of solvents selected in step (b) at elevated temperature, which is at least at or above said fractionation temperature, and wherein the extractive solvent/mixture of solvents to oil-blend ratio is from 1:2 to 100:1; (d) allowing a phase split to form between the heavy components fraction and the light components/extractive solvent fraction at the fractionation temperature or at most 10° C. below the fractionation temperature; (e) followed by separation of said fractions.

### FIELD OF THE INVENTION

The present invention relates to a process for liquid-liquid extraction of an oil-blend of non-uniform oligomers and polymers, in particular of a bio-oil. Further, the invention relates to a process for the production of a biofuel.

### BACKGROUND OF THE INVENTION

Lignocellulosic materials are of considerable interest as feedstocks for the production of sustainable biofuels as they may be converted into valuable intermediates, which intermediates may be further processed into fuel components.

Biofuels are combustible fuels, typically derived from biological sources, which result in a reduction of greenhouse gas emissions. Biofuels used for blending with conventional gasoline fuel components are alcohols, in particular ethanol. Biofuels such as fatty acid methyl esters derived from rapeseed and palm oil can be blended with conventional diesel components for use in diesel engines. However, these biofuels are derived from edible feedstock and so compete with food production.

Non-edible renewable feedstocks such as lignocellulosic biomass are therefore becoming increasingly important, both economically and environmentally, and there has been much interest in developing improved methods for producing useful compounds from such materials.

It is known that so-called “bio-oils” can be produced from lignocellulosic feedstock by several processes, e.g. by thermochemical conversion processes such as pyrolysis and liquefaction processes. Liquefaction processes may be performed at elevated temperatures and may involve the use of catalyst(s) and/or hydrogen. Direct liquefaction may be performed in (an) appropriate solvent(s), such as in a phenolic solvent like guaiacol.

Subsequently, the bio-oil may be processed in a conventional oil refinery for upgrading to transportation fuel. In liquefaction processes currently known in the art, at least part of the bio-oil can be recycled to be used as a liquefaction (co-)solvent in the process. However, the production of heavy components (molecular weight>1000 Da) was found to be a major hurdle in such processes as it prevents repeatedly recycling the bio-oil as liquefaction medium. Separating the heavy and light components in the bio-oil and recycling light components would solve that problem. Conventional distillation is a technique available for separation, but does not seem to be an economical and energy efficient separation technique. In the prior art the option that separation of liquefied products can be effected by liquid/liquid separation techniques has also been suggested, optionally in the presence of an extractive solvent. Another suggestion in the art was to effect separation of liquefied products products using a separation membrane followed by removal of any residual solvents by distillation. However, even though suggestions have been made for the use of certain techniques, an economical and energy efficient separation technique has not yet been reported. Therefore, there is a need for an economical and energy efficient process for separating the light components from the bio-oil product, for example to be able to run a liquefaction process in which at least a part of the bio-oil comprising light components is (continuously) recycled. Further, such a process is considered very advantageous for separating other types of mixtures or blends of non-uniform oligomeric and polymeric components such as mineral oils. A suitable extraction process is considered to be particularly attractive for blends that are not thermally stable and, thereby, tend to degrade during distillation at high temperature.

### SUMMARY OF THE INVENTION

The present invention provides a solution to that problem. It was found that liquid-liquid extraction can conveniently be used as a technique to separate the heavy and light components in a oil-blend of non-uniform oligomeric and polymeric components, in particular in a bio-oil, whichever the source of the bio-oil.

Accordingly, the present invention provides a process for liquid-liquid extraction of an oil-blend of non-uniform oligomeric and polymeric components, wherein “non-uniform” means that the components may have a varying size, shape and mass distribution, the process comprising a separation step wherein heavy components and light components in the oil-blend having similar chemical functionalities are separated to produce a heavy components fraction and a light components fraction, wherein the process comprises the steps (a) to (e):

(a) preselecting a desired molecular weight (Mw) boundary between heavy and light components;
(b) selecting an extractive solvent or an extractive mixture of solvents, which form essentially a single phase with the light components, such that at least 80% of the light components are dissolved, at elevated temperature, being the fractionation temperature, and in which the heavy components are essentially immiscible at the fractionation temperature, such that at most 10% of the heavy components are dissolved at said temperature in an amount of the extractive solvent/mixture of solvents in which the light components are fully dissolved at that temperature;
(c) mixing the oil-blend and the extractive solvent or extractive mixture of solvents selected in step (b) at elevated temperature, which is at least at or above said fractionation temperature, and wherein the extractive solvent/mixture of solvents to oil-blend ratio is from 1:2 to 100:1;
(d) allowing a phase split to form between the heavy components fraction and the light components/extractive solvent fraction at the fractionation temperature or at most 10° C. below the fractionation temperature;
(e) followed by separation of said fractions.

In an embodiment of the invention, the oil-blend of non-uniform oligomeric and polymeric components is a crude oil or a fraction thereof, which may contain very heavy crudes, tar sands and oil fractions. In a preferred embodiment said oil-blend is a bio-oil.

The term “non-uniform” herein means that the components may have a varying size, shape and mass distribution, wherein it is understood that oligomers and polymers may particularly possess a wide distribution range of molecular masses.

In an advantageous aspect of the invention, the process of this invention allows to use a temperature swing to recover the extractive solvent/mixture of solvents, which is expectedly more economical and energy efficient than the conventional distillation for solvent recovery.

Further, when used to separate light and heavy components from a bio-oil obtained from liquefaction processes, the process of the invention allows repetitive liquefaction runs with intermittent solvent extraction and recycle of the light components of the liquefied product stream.

The invention also provides a process for preparing a biofuel from the biocrude product(s), i.e. the heavy components fraction and/or the light components fraction, produced in a process comprising the liquid-liquid extraction process of the invention.

The process of the invention can be applied to separate the so-called ‘vacuum distillate’ fraction (in distillation terms this is the oil fraction that is distilled below 370-380° C. under vacuum) from the ‘vacuum residue’ fraction from various (bio-)oils to send the distillate fraction for processing to (bio)fuel (e.g. FCC or hydrocracking) without excessive coking that would otherwise result from the presence of the bituminous fraction. The vacuum residue fraction is a residue to be processed in a coker, Hycon, gasification unit to H₂/CO or as bitumen.

### BRIEF DESCRIPTION OF THE DRAWINGS

FIG. 1 shows a block diagram of liquid-liquid extraction. Major compounds present in different streams are shown between brackets.

FIG. 2 schematically shows (a) the prior art liquefaction process concept without fractionation of bio-oil and (b) the liquefaction process concept with fractionation of bio-oil according to the invention.

FIG. 3 depicts an experimental procedure of a multi-stage extraction followed by solvent recovery via temperature-swing. Legend: L: Lights, H: Heavies, S: Solvent.

FIG. 4 graphically shows the results of extraction of a light liquid effluent resulting from lignocellulose liquefaction with water/methanol and alkane solvents as extractive solvent/mixture of solvents. On the x-axis the extracted percentage is given and on the y-axis the vacuum residue fraction in the raffinate. Legend: between the brackets the water:methanol volume ratio is shown. Extraction temperatures: T=80° C. for alkanes, 70° C. for W/M(2:1), W/M(1.5:1) and 8° C. for W/M(0.67:1). Volume ratios: Solvent:Bio-oil=1:1, *Solvent:Bio-oil=2:1. The 100% selectivity line indicates a theoretical situation which assumes that all the vacuum residue (VR) lands in the raffinate, and no contamination of raffinate with the extraction solvent.

FIG. 5 graphically shows the results of multistage extraction: (a) VR fraction in the rejected stream versus the cumulative extracted percentage obtained in 4 stage extraction. (b) Distribution coefficient versus the cumulative extracted percentage. between the bracket the stage number is shown. Extractive solvent: water/methanol (3:2 vol. ratio), Extraction temperature: T=70° C., Recovery temperature: T=25° C.

FIG. 6 shows the VR fraction in reactor effluent versus cumulative wood (%) in the recycle experiments with (solid symbols) and without (open symbols) intermediate liquid-liquid extraction.

### DETAILED DESCRIPTION OF THE INVENTION

The direct liquefaction of lignocellulosic biomass using various solvents has been subject to investigations in recent years. It has been found that phenolic solvents are very suitable for liquefaction, such as guaiacol (very high liquid yield were obtained with guaiacol (>90 Co, see e.g. WO2013/072383). Guaiacol and its derivatives are produced by the liquefaction of biomass, which created the possibility to use a fraction of the resulting bio-oil as reaction medium by recycling it back to the liquefaction stage. Recycling initially succeeded in achieving high oil yield but readily lost its effectiveness as the liquid medium became very viscous because of the increasing formation of heavy products which slowly displaced the light start-up solvent (WO2013/072383). Further optimization of process parameters had limited effect on minimizing the formation of high molecular weight compounds (Industrial & Engineering Chemistry Research 2014, 53, (29), 11668-11676).

The present invention relates to separating the “light” components fraction (wherein suitably the boundary between heavy and light components being selected at molecular weight <1500 Da, preferably <1350 Da, more preferably <1250 Da, in particular <1100 Da, and especially, in this context, at molecular weight<1000 Da) from a bio-oil product and recycle that fraction back to the reactor to use that as a liquefaction solvent (see FIG. 2b). Generally, distillation is the most conventional process for fractionation of a homogeneous liquid mixture and it is widely used in refineries and chemical industries. The present invention however relates to liquid-liquid extraction, which—as a concept—is also used widely in the industry to separate a compound from a mixture of compounds. Extraction is based on the partial miscibility of compounds in the extractive solvent. Extraction works well when the compounds that need to be separated have different chemical functionalities, in particular polar and apolar and/or hydrogen donating or accepting functionalities. A chemical functionality herein is defined as a chemical functional group, which is a specific grouping of elements, being characteristic of a class of compounds, and which determines chemical properties and reactions of the that class. In liquid-liquid extraction, there is often a polar aqueous layer in which polar compounds dissolve and an apolar organic layer in which the apolar compounds dissolve.

However, generally bio-oils are mixtures of many components having a broad range in molecular weight, but all having similar chemical functionalities. In particular the components in bio-oil comprise aliphatic and aromatic structures and generally various oxygen-based functionalities throughout the whole molecular weight range. Thus, conventional liquid-liquid extraction using an apolar and a polar solvent will not be suitable for separation of heavy and light components in a bio-oil. While not wishing to be bound by theory, it is believed that the current process for liquid-liquid extraction of bio-oil to produce a light components fraction and a heavy components fraction, is an entropy-driven process. In terms of thermodynamics, this can be illustrated with the equation of change in Gibbs free energy at a certain temperature (T) and pressure (p) upon dissolving a given volume fraction in a solvent: ΔG(p,T)=ΔH−T*ΔS, in which ΔH is the change in enthalpy and ΔS the change in entropy. The change in enthalpy of dissolution for chemically similar compounds is comparable as the molecules present similar interactions with the solvent. The differentiating factor at a certain temperature is therefore the change in entropy, which is larger for a given volume fraction of many small molecules than of fewer large molecules. A person skilled in the art will recognize that the fractionation of light and heavy components in bio-oil is only feasible for solvents that show weak interaction with the oil-blend (i.e. with the solutes), preferably weak repulsive interactions. Solvents with too large “attractive” interaction AH will dissolve the heavy components in the bio-oil together with the light components while solvents with too large “repulsive” interaction with the oil-blend, will not dissolve the light components. The molecular weight of the molecules that dissolve in the solvent will vary with fractionation temperature—the lower the fractionation temperature, the lighter the molecules that are extracted. It is understood that the concept of this invention will not only be applicable to bio-oils, but that it will be equally applicable to other mixtures or blends of light and heavy components having similar chemical functionalities, such as, but not limited to, crude oil-blends.

Crude oil is a liquid petroleum, a type of hydrocarbon as it comes out of the ground, distinguished from refined oils manufactured out of it. The American Petroleum Institute, in its Manual of Petroleum Measurement Standards (MPMS), defines it as “a substance, generally liquid, occurring naturally in the earth and composed mainly of mixtures of chemical compounds of carbon and hydrogen with or without other nonmetallic elements such as sulfur, oxygen, and nitrogen.” It is liquid both in the subsurface and at standard surface conditions. Crude oil consists of a complex mixture of various hydrocarbons, largely of the alkane series, but may vary much in appearance and composition.

The selection of the extractive solvent/mixture of solvents according to the invention is done by selecting an extractive solvent/mixture of solvents which forms a single phase with the light components at the fractionation temperature, and in which the heavy components are essentially immiscible at that fractionation temperature, such that at most 10% of the heavy components are dissolved at said temperature in an amount of the extractive solvent/mixture of solvents in which the light components are essentially dissolved at that temperature such that at least 80%, preferably at least 90% and more preferably at least 95% of the light components are dissolved. The fractionation temperature may be selected and may be any suitable temperature that allows easy handling of the materials. Preferably, an extractive solvent/mixture of solvents is selected which has a fractionation temperature with the light components of the bio-oil that is above 0° C., more preferably above 10° C., more preferred above 30° C., in particular preferred above 40° C., and especially above 60° C., suitably up to 250° C., preferably up to 200° C., more preferably up to 150° C., even more preferred up to 120° C., and especially up to 100° C.

The process of the invention is a specific liquid-liquid extraction process which was found to be less energy demanding than distillation. The use of a weakly interacting solvent allows to use a temperature swing (thus a difference in temperatures) to efficiently recover the extractive solvent/mixture of solvents by spontaneous liquid-liquid separation of the light bio-oil from the extraction solvent upon cooling. One of the major energy consuming steps in extraction is the recovery of the extractive solvent, which is generally done by an energy intensive stripping or distillation process. It was found that a more economical way of recovering the solvent is the use of a temperature swing. In order to utilize temperature swing for recovery of the extractive solvent/mixture of solvents according to the process of the present invention, the extractive solvent/mixture of solvents is able to form two phases with the liquefied product stream at low temperature (say room temperature) and mixes at moderately high temperature (i.e. above fractionation temperature).

Accordingly, in a further embodiment of the invention preferably the extractive solvent/mixture of solvents in step (b) is selected such that it not only forms a single phase with the light components at the fractionation temperature, but that it also demixes from the light components at a lower temperature, being the demixing temperature and wherein the separation step (e) is followed by cooling of the light components/extractive solvent fraction to the demixing temperature or lower and allowing demixing thereof, subsequently followed by a further separation step (f) to recover a light components stream and the extractive solvent/mixture of solvents.

One of the measures of the polarity of a solvent is its log P value, where P is defined as the partition coefficient of a compound in a two phase octanol-water system. The log P value can be determined experimentally or calculated according to standard procedures as discussed e.g. in Handbook of Chemistry and Physics, 83^{rd} Edition, pages 16-43 to 16-47, CRC Press (2002), and see also J.Phys.Chem.Ref. Data 1989, 18 and VCClab (http://www.vcclab.org/lab/alogps/).

The Hildebrand and Hansen solubility parameters are other measures that can be used for the polarity of a solvent. The Hildebrand solubility parameter (6) provides a numerical estimate of the degree of interaction between materials, and can be a good indication of solubility, particularly for nonpolar materials such as many polymers. Materials with similar values of δ are likely to be miscible. The Hildebrand solubility parameter is the square root of the cohesive energy density, which is the amount of energy needed to completely remove unit volume of molecules from their neighbours to infinite separation (an ideal gas). This is equal to the heat of vaporization of the compound divided by its molar volume in the condensed phase. In order for a material to dissolve, these same interactions need to be overcome as the molecules are separated from each other and surrounded by the solvent. Dr. J. H. Hildebrand suggested the square root of the cohesive energy density as a numerical value indicating solvency behavior. This later became known as the “Hildebrand solubility parameter”. Materials with similar solubility parameters will be able to interact with each other, resulting in solvation, miscibility or swelling. Regarding the Hansen parameter, the three Hansen parameters (dispersion forces, dipolar intermolecular forces and hydrogen bonds) can be treated as co-ordinates for a point in three dimensions also known as the Hansen space. The nearer two molecules are in this three-dimensional space, the more likely they are to dissolve into each other. To determine if the parameters of two molecules (usually a solvent and a polymer) are within range a value called interaction radius (Rₐ) is given to the substance being dissolved. This value determines the radius of the sphere in Hansen space and its center is the three Hansen parameters.

As for liquid-liquid extraction of bio-oil that largely consist of phenolic and/or furanic structure, as an embodiment of the present invention, solvents or solvent mixtures with low polarity, that is in particular log P>1.0, are suitable solvents for a process in which a temperature swing is used. For instance, such solvents are immiscible with guaiacol at room temperature and are miscible at higher temperature. Guaiacol is a relevant model compound for the light components in the liquefaction bio-oil. In particular, extractive solvents/mixtures of solvents having a low polarity with log P>1.0, particularly up to log P=10, are suitable, and preferably log P>2.0, more preferred log P>2.5, even more preferred log P>3.0, and highly preferred log P>3.5. In particular preferred solvents/extractive mixture of solvents have a log P>3.0 and are selected from C6-C16 (cyclo)alkanes or mixtures thereof, and a particularly useful solvent is hexadecane.

According to another embodiment of the present invention, also solvents or solvent mixtures with high polarity may be used as suitable solvents for the process according to the invention in which a temperature swing is used. For solvents or solvent mixtures with high polarity, the Hildebrand solubility parameter is used, as for example for water the partition coefficient log P is not defined and should be infinitely negative. The Hildebrand solubility parameter provides a numerical estimate of the degree of interaction between materials, and is a good indication of solubility. Accordingly, solvents or solvent mixtures with high polarity, preferably that is with a Hildebrand solubility parameter above 37 [J/ml]^{0.5} and, preferably from 37 up to 41 [J/ml]^{0.5}, are suitable solvents for the process of extraction light liquefaction bio-oil according to the invention in which a temperature swing is used. In particular, it was found that water-methanol mixtures are very suitable as extractive mixture of solvents, preferably in the volume ratio of 1:2 to 2:1, and especially in a volume ratio of 3:2.

These solvents/solvent mixtures with either low polarity, in particular with log P>3.0, or high polarity, in particular with Hildebrand solubility parameter from 37 [J/ml]^{0.5}, particularly up to 41 [J/ml]^{0.5} show affinity towards the light components in the bio-oil at elevated temperature (>40° C.) but appear to demix from the light bio-oil at lower temperature.

In a further embodiment of the invention, solvents/solvent mixtures with Hansen interaction radius Ra>16 are generally very suitable for the process of this invention for liquid-liquid extraction of an oil-blend of non-uniform oligomeric and polymeric components, in particular oil-blends comprising bio-oil and/or crude oil.

The extractive solvent/mixture of solvents to oil-blend ratio used in the process of this invention is from 1:2 to 100:1, preferably from 1:2 to 10:1, in particular from 1:1 to 5:1. Very suitably, the ratio is from 1:1 to 2:1, wherein the ratio is measured in volume.

The process of the present invention is in particular useful for the separation of bio-oil obtained in liquefaction processes. Accordingly, an embodiment of the present invention relates to a process for liquefying a cellulosic material comprising: a step (i) wherein the cellulosic material is contacted with a solvent or mixture of solvents to produce a liquefied product stream, and a recycle step (ii) in which at least a part of the liquefied product stream is recycled to step (i), wherein the process comprises the above described process for liquid-liquid extraction to extract the liquefied product stream, wherein at least a part of the light components fraction obtained in step (e) is recycled to step (i) as at least a part of the liquefied product stream that is recycled in step (ii). In another embodiment of the invention, a temperature swing is used for further separation of the extractive solvent(s) and the light components stream, allowing use of the latter in the liquefaction. Accordingly, the separation step (e) is followed by cooling of the light components fraction and demixing thereof, subsequently followed by a further step to recover a light components stream and the extractive solvent/mixture of solvents and wherein at least a part of the light components stream is recycled to step (i). In still another embodiment, the separation step (e) is followed by cooling of the light components fraction and demixing thereof, subsequently followed by a further step to recover a light components stream and the extractive solvent/mixture of solvents and wherein the process comprises a step in which the extractive solvent/mixture of solvents is recycled to step (i) to be re-used as liquefaction solvent, which is particularly useful when the extractive solvent/mixture of solvents is Light Cycle Oil (LCO). In an embodiment of the invention, the solvent in step (i) and the extractive solvent/mixture of solvents is the same solvent and is LCO. Suitably, the recovered extractive solvent/mixture of solvents may also be re-used to further extract the heavy components fraction. In a further embodiment, e.g. for process efficiency reasons, the process may comprise a subsequent distillation step to recover light components from the heavy components fraction.

The extraction may be performed in different flow modes, selected from co-current, cross-flow and counter-current extraction. Further, the extraction may be performed in a single step, but also suitably as multistep extraction. Extraction equipment may be selected from any suitable commercially available equipment, such as extraction equipment using empty vessels, vessels with static internals such as trays or packings (random or structured), vessels with moving internals such as rotating or oscillating disks or rotating propeller, or any combination thereof.

By liquefying is herein understood the conversion of a solid material, such as cellulosic material, into one or more liquefied products. Liquefying is sometimes also referred to as liquefaction.

By a liquefied product is herein understood a product that is liquid at ambient temperature (20° C.) and pressure (1 bar absolute) and/or a product that can be converted into a liquid by melting (for example by applying heat) or dissolving in a solvent. Preferably the liquefied product is liquid at ambient temperature (20° C.) and pressure (1 bar absolute).

Liquefaction of a cellulosic material can comprise cleavage of covalent linkages in that cellulosic material. For example liquefaction of lignocellulosic material can comprise cleavage of covalent linkages in the cellulose, hemicellulose and lignin present and/or cleavage of covalent linkages between lignin, hemicelluloses and/or cellulose.

As used herein, cellulosic material refers to material containing cellulose. Preferably the cellulosic material is a lignocellulosic material comprising lignin, cellulose and optionally hemicellulose.

Any suitable cellulose-containing material may be used in the process according to the present invention. Advantageously, cellulosic material for use according to the invention may be obtained from a variety of plants and plant materials including agricultural wastes, forestry wastes and sugar processing residues. Examples of suitable cellulose-containing materials include agricultural wastes such as corn stover, soybean stover, corn cobs, rice straw, rice hulls, oat hulls, corn fiber, cereal straws such as wheat, barley, rye and oat straw; grasses; forestry products such as wood and wood-related materials such as sawdust; waste paper; sugar processing residues such as bagasse and beet pulp; or mixtures thereof.

Before being used in the process of the invention, the cellulosic material is preferably comminuted into small pieces in order to facilitate liquefaction. Conveniently, the lignocellulosic material can be comminuted into pieces of average length of 0.5 to 30 mm.

Any light components fraction obtainable from the cellulosic material liquefied according to the process of the invention may advantageously be recycled and used as a (make-up) solvent in the liquefaction process, affording significant economic and processing advantages.

In a preferred embodiment the recycled light components fraction comprises a weight amount of solvent (mixture) of 2 to 100 times the weight of the cellulosic material, more preferably of 5 to 20 times the weight of the cellulosic material.

For liquefaction, the liquefaction solvent/mixture of solvents may be selected from water, oxygenates such alcohols, ketones and phenolic components, a fraction of the bio-oil produced by the liquefaction process or a hydrocarbon stream such as present in oil refineries. In one embodiment, the liquefaction solvent/mixture of solvents is the light fraction of the bio-oil that is recycled after extraction with the extractive solvent. In another embodiment, the extractive solvent is used as liquefaction solvent and recycled while the light and heavy bio-oil are both recovered for further upgrading, e.g. in an oil refinery. A suitable solvent for that purpose is a hydrocarbon refinery stream such as Light Cycle Oil or vacuum gasoil.

In the liquefaction process, the cellulosic material and the liquefaction solvent/mixture of solvents, are preferably mixed in a liquefaction solvent (mixture)-to-cellulosic material ratio of 2:1 to 20:1 by weight, more preferably in a solvent (mixture)-to-cellulosic material ratio of 3:1 to 15:1 by weight and most preferably in a liquefaction solvent (mixture)-to-cellulosic material ratio of 4:1 to 10:1 by weight.

The liquefaction process according to the invention is preferably carried out at a temperature of from 100° C. to 450° C. More preferably, the process is carried out at a temperature of from 250° C. to 380° C., most preferably from 280° C. to 350° C.

Preferably the liquefaction process is performed under autogeneous pressure.

Each of the light and heavy components fractions obtained after the liquid-liquid extraction of the present invention may be transferred for subsequent conversion to biofuels in separate treatment routes.

As used herein, a biofuel is a component or mixture of components that is derived from biomass and can be used as a fuel or fuel component.

In an embodiment, the products of the process of the invention and optionally hydrogenated products derivable therefrom may be converted into biofuels.

Suitably, the products of the process this invention or the hydrogenated products derivable therefrom may be converted to biofuels using techniques such as hydro-deoxygenation or thermal-, catalytic- or hydro-cracking processes.

In one embodiment, the optionally stabilized light and/or heavy bio-oil fractions products are at least partially hydrodeoxygenated, rendering them hydrocarbon soluble, prior to being blended with a refinery stream such as crude oil, (vacuum) gasoil or (heavy) cycle oil and being subjected to further hydrodeoxygenation or a thermal-, catalytic- or hydro-cracking processes.

Suitably, the hydrodeoxygenation may be performed under conditions in the presence of a supported heterogenous metal or metal sulfide catalyst. The metal catalyst suitably comprises a metal of any one of groups 8 to 11 of the Periodic Table of Elements such as iron, cobalt, nickel, ruthenium, rhodium, palladium, iridium or platinum. Metal sulfide catalysts suitably comprise sulfided molybdenum optionally promoted with cobalt or nickel.

Following an initial hydrodeoxygenation step, the at least partially deoxygenated biocrude products can be recovered from the solvents, by conventional separation techniques, prior to being subjected to upgrading to hydrocarbons by means of further hydrodeoxygenation or by thermal-,catalytic- or hydro-cracking processes.

Throughout the description and claims of this specification, the words “comprise” and “contain” and variations of the words, for example “comprising” and “comprises”, mean “including but not limited to”, and do not exclude other moieties, additives, components, integers or steps.

### Definitions Used

“Liquefaction solvent”: A compound used for liquefying wood particles. In the process presently described, guaiacol is used as a starting liquefaction solvent. However in a continuous operation, it will be eventually replaced by light bio-oil.

“Bio-oil”: Any product stream obtained from either pyrolysis or liquefaction processes, for example being a liquefied product stream obtained after liquefaction of wood which stream consists of single pass reactor products and, optionally, the liquefaction solvent when used. According to the present invention “bio-oil” is further divided into two fractions based on apparent molecular weight (as determined by GPC referred below as M_{W,GPC}): lights and heavies, see below.

“Lights/Light bio-oil”: the bio-oil with preferably M_{W,GPC}<1000 Da. Molar mass cut-off is arbitrarily chosen to 1000 Da.

“Heavies/Heavy residue” or “Vacuum Residue(VR)”: the the bio-oil with preferably M_{W,GPC}>1000 Da.

“Vacuum residue fraction”: is defined as the fraction of the bio-oil that consists of vacuum residue (M_{W,GPC}>1000 Da—see above) based on equation 1. It is conveniently determined by means of GPC assuming for instance comparable response factor for the light and heavy bio-oil components.

$$Vacuum residue fraction = \frac{Area corresponds to {M}_{W , GPC} > 1000 Da}{Total GPC Area} \qquad ( equation 1 )$$

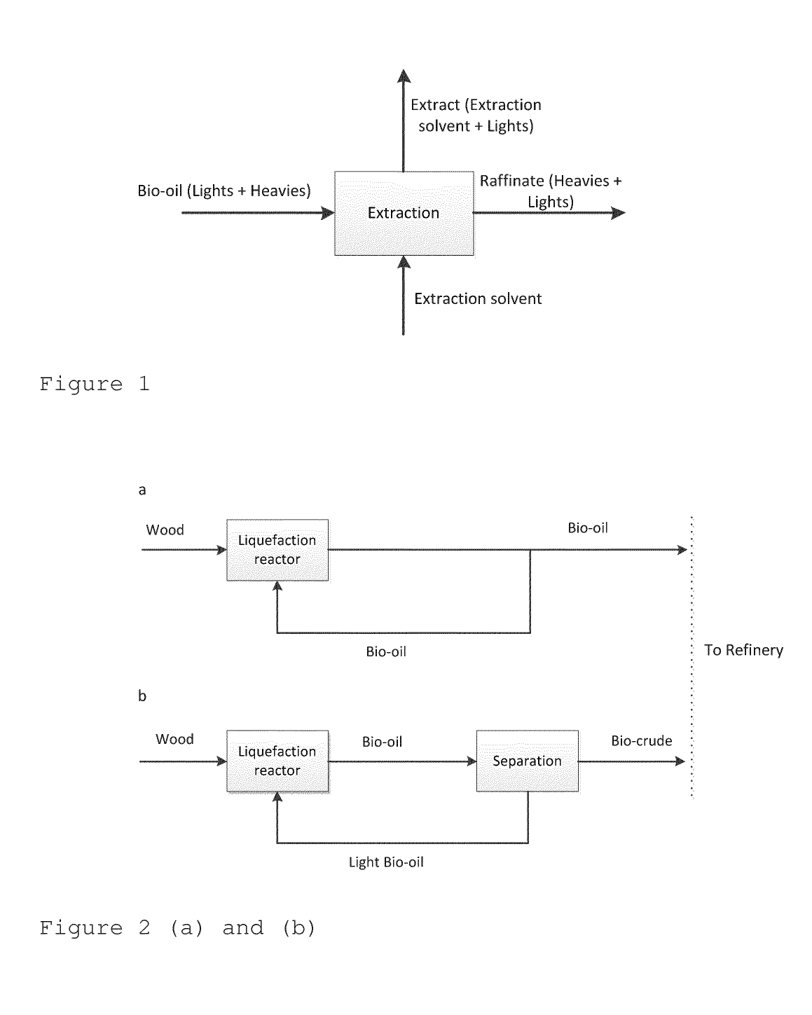

“Extracted percentage”: A term used to characterize liquid-liquid extraction (see FIG. 1) which is the percentage of the feed (bio-oil) that is extracted by the extraction solvent.

“Cumulative wood concentration”: this represents the wood fraction of the total fresh intake, i.e. fresh wood and fresh/make-up liquefaction solvent. It is calculated from the amount of fresh wood used in a given refill run, the amount of recycle solvent and its cumulative wood concentration accumulated over all previous recycle runs and the amount of fresh solvent used in a specific run (equation 2). A 100% cumulative wood means that the liquefaction solvent is purely derived from wood.

$$( equation 2 )$$

$$Cumulative wood ( w \% ) = \frac{Cummulative wood intake [ g ]}{Cum . wood intake [ g ] + Fresh solvent intake [ g ]} \times 100 = \frac{\begin{matrix}Fresh wood [ g ] + Recycle solvent [ g ] \times \\ Cum . wood concentration of recycle solvent\end{matrix}}{Fresh wood [ g ] + Recycle solvent [ g ] + Fresh solvent [ g ]} \times 100$$

“Distribution coefficient”: is defined as ratio of solute concentration in the extract and solute concentration in the raffinate (equation 3). Generally concentration is defined as mol/liter however here it is defined as kg/kg for convenience. Here solute is light bio-oil.

$$Distribution coefficient = \frac{\frac{( Mass of light bio \text{-} oil in extract )}{( Mass of total extract )}}{\frac{( Mass of light bio \text{-} oil in raffinate )}{( Mass of total raffinate )}} \qquad ( equation 3 )$$

### DETAILED DESCRIPTION OF SOME DRAWINGS

FIG. 1 shows a scheme of liquid-liquid extraction and nomenclature of various streams and their main constituents. A low rejection is desired which will result in higher extraction of the lights. Ideally, all the heavies should land in the raffinate stream and the lights should go in the extract stream. This means for example that single pass reactor products of liquefaction processes (i.e. solvent lean bio-oil) ideally land in the raffinate and the rest should go in the extract.

FIG. 2b shows process concept investigated in this work while FIG. 2a shows the prior art process concept which resulted in build-up of heavies and subsequent increase in medium viscosity (WO2013/072383).

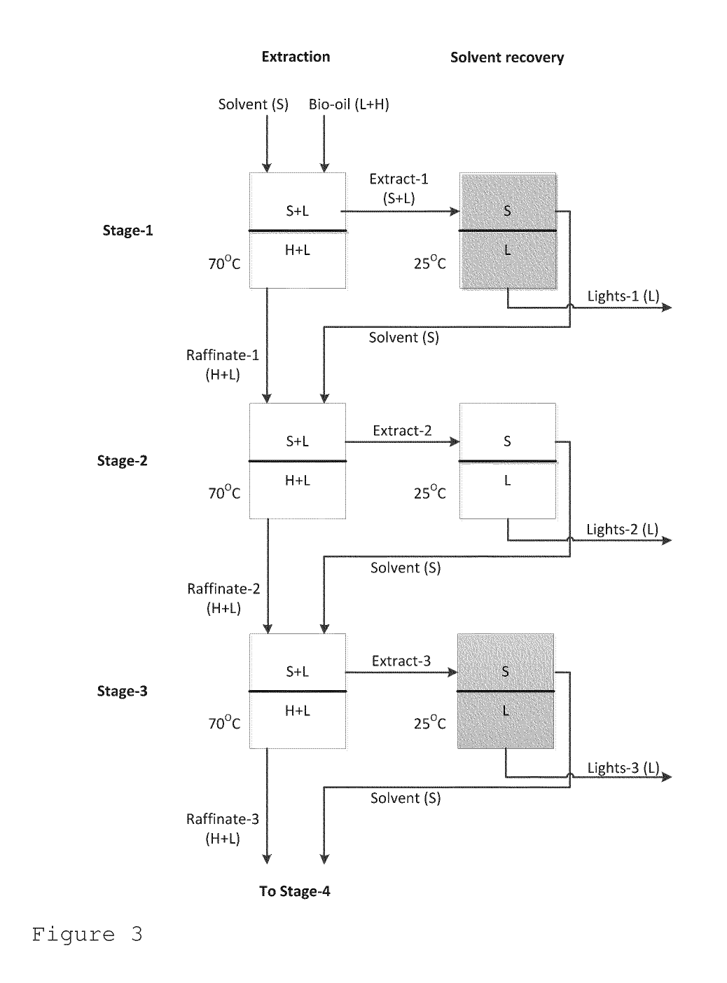

In FIG. 3 the procedure of multistage liquid-liquid extraction is shown and can be described as follows: the bio-oil is mixed with a suitable extractive solvent. While being stirred, the mixture is heated to the fractionation temperature or a bit higher, and kept at that temperature for some time to allow intense mixing. Then the phases are allowed to split below the fractionation temperature. The extract (rich in light bio-oil (L)) is allowed to cool down to room temperature resulting in two phases, a solvent phase and a light bio-oil phase. The raffinate (rich in heavy bio-oil (H)) is further subjected to a second stage extraction using the solvent regenerated in the previous stage. This is then followed by a cooling stage to recover the solvent, and subsequent extraction steps, e.g. a 3^{rd} and 4^{th} extraction step.

### Examples

The invention will now be further illustrated by means of the following non-limiting examples and comparative examples.

### Abbreviations

### Da Dalton

### GPC Gel Permeable Chromatography

M_{W,GPC} Molecular weight defined by GPC
M_{W} Molecular weight
RID Refractive index detector

### T Temperature

VR Vacuum residue=heavy bio-oil

Liquid-Liquid extraction was studied to explore the possibility to recover the light bio-oil for recycling as liquefaction solvent. First, a screening of various potential solvents was carried out by checking their immiscibility with guaiacol as a model for the bio-oil. The most promising solvents were then evaluated with real liquefaction product. Experiments were designed to determine the fractionation temperature of various extractive solvents. In case of extraction of real bio-oil, an increase in temperature above fractionation temperature would result in extraction of light components which can be separated back by cooling the mixture below the fractionation temperature, which was investigated experimentally. Finally a proof of concept was provided experimentally by carrying out multiple liquefaction runs with inter-stage recovery and recycle of the light components as liquefaction solvent.

### Materials and Methods

### Materials

Pine wood was obtained from Rettenmaier & Sohne GmbH (Germany). It was crushed to the particle size of <0.5 mm and then was dried at 105° C. for 24 hours in an oven. The composition of the pine wood is provided in Table 1 (see also Industrial & Engineering Chemistry Research 2007, 46, (26), 9238-9247). All other chemicals were obtained from Sigma Aldrich with a purity >98%.

TABLE 1
Pine wood composition

| Composition | Value |
|---|---|
| chemical analysis | wt. %, dry |
| cellulose | 35 |
| hemicellulose | 29 |
| lignin | 28 |
| alkali metals | mg/kg, dry |
| K | 34 |
| Mg | 134 |
| Ca | 768 |
| total ash | 2600 |
| ultimate analysis | wt. %, daf |
| C | 46.58 |
| H | 6.34 |
| O (by difference) | 46.98 |
| N | 0.04 |
| S | 0.06 |

A bio-oil was produced in house by conducting a liquefaction experiment with feed composition as shown in Table 2. The extraction experiment was carried out using this bio-oil.

TABLE 2
Feed and process conditions used in liquefaction of
pine wood.

| Feed | wt. % |
|---|---|
| Guaiacol | 55 |
| Wood | 30 |
| Water | 15 |
| Autoclave volume (L) | 0.56 L |
| Pressure (bar) | 90 |
| Temperature (° C.) | 300 |
| Reaction time (min) | 100 |

### Experimental Set-Up and Procedure

### Liquid-Liquid Extraction

Extraction was carried out in a laboratory glass beaker. Heating was done using a temperature controller electrical heating plate and mixing was done using a magnetic stirrer. Extraction experiments were carried out in two steps. Firstly, several compounds were screened for weak interaction with guaiacol, which was selected as model compound for bio-oil. Then, the promising solvents were evaluated for extraction of light bio-oil out of an actual liquefaction product. For the liquid-liquid extraction, the bio-oil produced according to Table 2 was used.

### Example 1: Screening of Solvents

Initial work was done to screen for a suitable extraction solvent to extract lighter compounds and reject heavier compounds. The experimental procedure was as follows: equal volumes of the model compound guaiacol and the solvent were mixed and stirred using a magnetic stirrer at room temperature. The minimum temperature needed for Liquid/Liquid (L/L) mixing was then determined by heating the mixture. Those solvents which were capable of phase splitting at room temperature and able to form a single phase at elevated temperature, were further tested with the bio-oil.

The experimental procedure with the bio-oil was as follows: extraction solvent and the bio-oil were mixed in a fixed volume ratio and stirred using a magnetic stirrer. The mixture was heated to 10 to 30° C. above the fractionation temperature to increase amount of extracted bio-oil. The stirrer was turned off and the mixture was allowed to settle down for a phase split below the fractionation temperature. The resulting two phases, being a heavy bio-oil phase and a light bio-oil/extractive solvent phase, were separated out using a syringe and further cooled to room temperature, which led to phase split of the extracted light bio-oil and the extractive solvent. Both the extracted light bio-oil (phase split after cooling) and the remaining heavy bio-oil after solvent extraction (raffinate), were analyzed in GPC.

Results:

Organic compounds with moderate polarity, that is with a water-octanol partition coefficient log P <3.1, form a single phase with guaiacol even at −20° C. In contrast, compounds with low polarity, that is in particular when log P >3.1, are immiscible with guaiacol at room temperature but become miscible at high temperature (Table 3).

TABLE 3
Screening of solvents - Extraction test results with guaiacol.

| | | | Hansen [J/ml]^{0.5} | | | | | |
|---|---|---|---|---|---|---|---|---|
| | Transition | Hildebrand | | | H- | Ra (vs | log P | log P |
| | at (° C.) | [J/ml]^{0.5} | Dispersion | Polar | Bonding | Guaiacol) | (VCClab) | (Sangster) |
| Water | 180 | 47.8 | 15.5 | 16 | 42.3 | 28.8 | | |
| Water/Methanol | 40 | 40.5 | 15.34 | 14.52 | 34.3 | 20.7 | | |
| (60:40) | | | | | | | | |
| Water/Methanol | 5 | 38.7 | 15.3 | 14.15 | 32.3 | 18.7 | | |
| (50:50) | | | | | | | | |
| Glycerol | X | 36.2 | 17.4 | 12.1 | 29.3 | 15.3 | −1.58 | −1.74 |
| Methanol | X | 29.6 | 15.1 | 12.3 | 22.3 | 8.9 | −0.59 | −0.74 |
| Ethanol | X | 26.5 | 15.8 | 8.8 | 19.4 | 5.0 | −0.07 | −0.3 |
| Furfural | X | 24.4 | 18.6 | 14.9 | 5.1 | 12.2 | 0.53 | 0.46 |
| guaiacol | X | 23.5 | 16.3 | 8.7 | 14.5 | 0.0 | 1.41 | 1.32 |
| n-butanol | X | 23.2 | 16 | 5.7 | 15.8 | 3.3 | 0.93 | 0.84 |
| Acetic acid | X | 21.4 | 14.5 | 8 | 13.5 | 3.8 | −0.13 | −0.17 |
| 1-Me- | X | 21.1 | 20.6 | 0.8 | 4.7 | 15.2 | 3.63 | 3.87 |
| naphtalene | | | | | | | | |
| 1-octanol | X | 20.6 | 16 | 5 | 11.9 | 4.6 | 3.05 | 3.07 |
| Toluene | X | 18.2 | 18 | 1.4 | 2 | 14.9 | 2.5 | 2.73 |
| Ethylbenzene | X | 17.9 | 17.8 | 0.6 | 1.4 | 15.7 | 2.96 | 3.15 |
| Cyclohexane | 11 | 16.8 | 16.8 | 0 | 0.2 | 16.8 | 3.12 | 3.44 |
| n-Hexadecane | 67 | 16.3 | 16.3 | 0 | 0 | 16.9 | 7.83 | |
| n-Undecane | 54 | 16 | 16 | 0 | 0 | 16.9 | 5.52 | 6.54 |
| n-Dodecane | 58 | 16 | 16 | 0 | 0 | 16.9 | 5.99 | 6.1 |
| diethyl ether | X | 15.6 | 14.5 | 2.9 | 5.1 | 11.6 | 0.97 | 0.89 |
| n-Octane | 48 | 15.5 | 15.5 | 0 | 0 | 17.0 | 4.36 | 5.15 |
| n-Heptane | 47 | 15.3 | 15.3 | 0 | 0 | 17.0 | 4 | 4.66 |
| n-Hexane | 41 | 14.9 | 14.9 | 0 | 0 | 17.1 | 3.55 | 3.9 |

Interestingly, very polar media such as water and water/methanol mixtures were also immiscible at low temperature and miscible at high temperature. Partition coefficient (Octanol-water partition coefficient) of the tested solvents were obtained from Sangster (J.Phys.Chem.Ref.Data 1989, 18) and using VCClab (http://www.vcclab.org/lab/alogps/). The partition coefficient of water is not defined and should be infinitely negative.

This screening showed that a solvent with either a high or a very low/negative log P is required for hot solubility and cold immiscibility of guaiacol and, expectedly, of the light bio-oil.

In Table 3 also the Hildebrand and Hansen parameters of the tested solvents are listed. The Hildebrand solubility parameter provides a numerical estimate of the degree of interaction between materials, and can be a good indication of solubility. For highly polar media this parameter is more useful than log P to define solubility properties.

It can further be seen in Table 3 that solvents (mixtures) with a Hansen interaction radius Ra>16 are suitable solvents, all forming two liquid phases at room temperature but a single phase at elevated temperature.

### Example 2: Extraction of Bio-Oil

The solvent systems that showed immiscibility at room temperature and mixing at higher temperature were further tested with a bio-oil, namely several water/methanol mixtures as well as some alkanes. These solvent systems showed significant selectivity for extracting the lighter components above the guaiacol fractionation temperature. The liquid-liquid phase split resulted then in an extract that was rich in extractive solvent and light bio-oil, and a raffinate that was rich in heavy bio-oil and low in extractive solvent and low in light bio-oil. Moreover, the extractive solvent could then be recovered from the light bio-oil upon cooling below guaiacol/solvent fractionation temperature. However in this work it was cooled to room temperature for convenience.

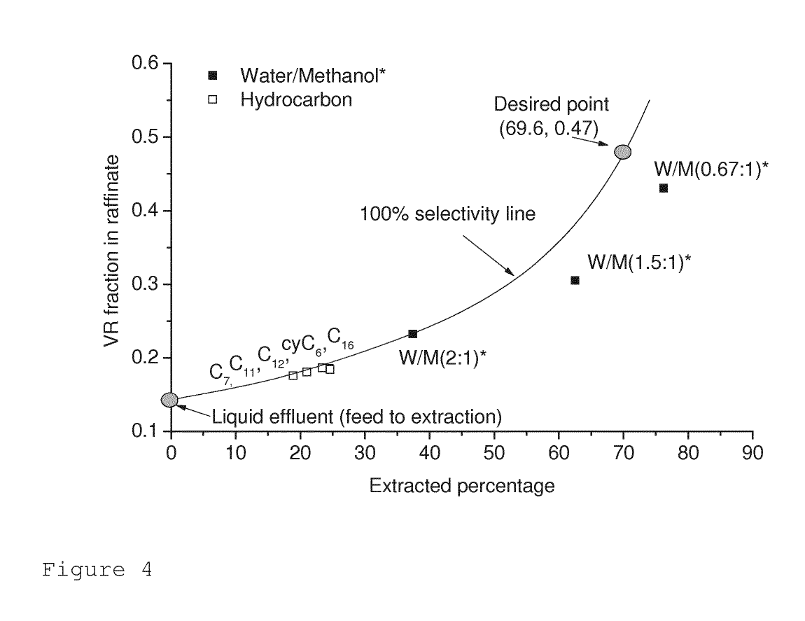

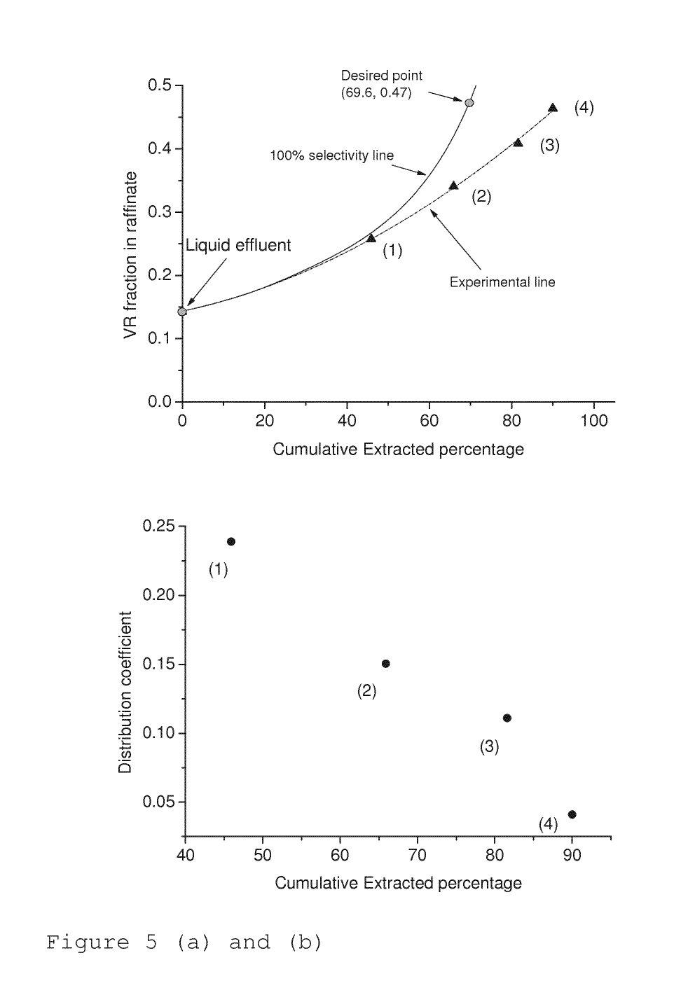

FIG. 4 shows a comparison of all studied solvents and shows a general increase in Vacuum Residue (VR) fraction in the raffinate with increasing extracted percentage. Here, the liquid effluent fed to the extraction is reported as 0% of extracted percentage and VR fraction of 0.143. The extraction process seems to proceed with nearly 100% selectivity at low extracted percentage but becomes somewhat less selective at higher extracted percentage, since the experimental data lie below the 100%-selectivity line that stretches between the feed point at (0, 0.143) and the ideal point of fully selective bio-crude fractionation of (85.7, 1) represented by the solid line in FIG. 4. It should be noted here that 100% selectivity line cannot go beyond extracted percentage of 100-14.3 (percentage of heavy liquid effluent in the feed)=85.7, that is till the extraction of all the light liquid effluent, which will result in VR fraction equal to 1 in the raffinate stream. The calculated desired point of (69.6,0.47) corresponds to the 100% selective extraction that is required to recover 95% of the solvent for recycling to the liquefaction at solvent:wood:water ratio of 55:30:15. Water/Methanol mixtures give high extracted percentages and a raffinate concentrated with VR while hydrocarbons give low extracted percentages with low VR in the raffinate. Slight deviation of experimental points from the 100% selectivity line is possibly due to presence of extractive solvent in the raffinate stream, giving lower VR. Noteworthy, the mixture with W/M(0.67:1)* did not show a clear phase separation even at room temperature and hence it was cooled down to 8° C. for a clear phase separation. Also in this case, the extractive solvent could not be separated by just cooling to room temperature (needed to be cooled below 8° C.) unlike in other cases where extracted solvent was recovered by cooling to room temperature. The W/M(1.5:1)* mixture and hexadecane seem the most promising solvents among the W/M mixtures and alkanes, respectively, as they give relatively high extracted percentages in a single stage liquid-liquid extraction. The extracted percentage can be further increased by applying multi stage extraction. These two solvents were further explored.

### Example 3: Multistage Extraction

An extractive solvent (S) was prepared by mixing water and methanol in 3:2 volume ratio. The experimental procedure of multistage liquid-liquid extraction is shown in FIG. 1 and can be described as follows: the bio-oil obtained from the liquefaction experiment was mixed with extractive solvent (mixture of water and methanol) in the volume ratio of 1:2. The mixture was stirred with a magnetic stirrer and heated to extraction temperature of 70° C. It was stirred for around half an hour at the extraction temperature and then phases were allowed to split. The extract was rich in light bio-oil (L) and the raffinate was rich in heavy bio-oil (H). The extract was cooled down to room temperature, which resulted in two phases, a solvent phase and a light bio-oil phase. The raffinate was further subjected to a second stage extraction using the solvent regenerated in the previous stage. This was followed by a cooling stage to recover the solvent and a 3^{rd} and 4^{th} extraction step.

Results: Bio-oil was subjected to a multi stage extraction using water/methanol mixture (1.5:1). Accordingly, the VR-rich raffinate of the first stage was subjected to three consecutive extractions using the same water/methanol that was purified by cooling down to room temperature and liquid-liquid separation of the extractive solvent and extracted oil. FIG. 5a shows cumulative extracted percentages versus VR in the rejected stream. The experimental extracted percentage was calculated based on mass of the raffinate stream. The 100% selectivity line assumes that all the VR lands in the raffinate stream without contamination of raffinate with the extractive solvent. However in practice, the fractionation is not fully selective.

After four stages, the cumulative extracted percentage was increased to 90 w %. However, it reached already 66 w % after stage 2, which corresponds to a recovery of 95% of the solvent required for liquefaction with solvent:wood:water ratio of 55:30:15. That means that a slight adjustment in process parameters can result in a recovery of liquefaction solvent of 100% in two stages only. So for a process, two extraction stages are sufficient to obtain a required amount of extracted liquid effluent needed to close the recycle loop. A loss of around 20 wt. % of the extractive solvent was observed in the extraction runs. The water content of the solvent recovered after 4^{th} stage matches the initial water content, which means that the loss was not selective to either water or methanol. Loss of solvent was probably due to evaporation as well as dissolution of the solvent in the oil phase. However the amount of methanol diffused in the oil phase was not measured. The distribution coefficient of the light liquid effluent decreases sharply with increase in cumulative extracted percentage (FIG. 5b). That means that more solvent will be needed to achieve the same recovery at higher extracted percentage. Therefore it seems not wise to go for a very high extracted percentage (deep recovery of light liquid effluent) using liquid-liquid extraction. For a process point of view, it would be beneficial to carry out extraction at low extracted percentage and then use distillation to recover the rest required amount of the light bio-oil.

GPC analysis of extracted light bio-oils isolated after solvent recovery had similar Mw distribution in all the four stages, with guaiacol at 100 Da and light bio-oil centered around 300 Da. The raffinates showed slight enrichment of vacuum residue and small shift towards higher Mw with increasing number of extraction stages. Here also no improvement in raffinate-4 from raffinate-3 could be seen. The recovered extraction solvent after stage-4 contained guaiacol in amounts that did not change significantly over the 4 stages. Water/methanol could not be detected in the GPC.

The final raffinate was still fairly rich in light bio-oil, including guaiacol. The VR enrichment after three extraction stages was about a factor three (VR fraction from 0.14 to 0.47). Further enrichment would require more extraction stages but might be achieved more effectively by means of distillation of the raffinate.

### Example 4: Liquefaction with Bio-Oil Extraction and Recycle

The concept of solvent extraction and recycle was further investigated by performing several consecutive liquefaction experiments with intermittent extraction and recycle of the liquefaction solvent. First, a liquefaction experiment was carried out at 320° C., in a 45 mL batch autoclave using a feed of guaiacol:wood:water of 55:30:15 in weight ratio. Experimental set-up and procedure for liquefaction of wood is as described in Industrial & Engineering Chemistry Research 2014, 53, (29), 11668-11676. The resulting liquid product was subjected to a 2-stage extraction with a 5-fold volume of hexadecane following a procedure similar to that described above and in FIG. 3. Namely, the mixture was stirred for half an hour at 90° C. and then let unstirred for settling down two phases, which were separated into an extract phase and a raffinate phase. The extract cooled down to allow phase separation of the extracted light bio-oil from nearly pure hexadecane. The raffinate was subjected to a second stage extraction using the solvent regenerated in the first stage, following the same procedure as used in the first stage. The light bio-oil recovered in the first and second stage were mixed together and used as a liquefaction solvent for second liquefaction run and a second extraction step. This procedure was followed for 5 liquefaction runs and subsequent extraction steps. Some fresh guaiacol was added to the second liquefaction run only in order to make-up for the loss incurred to saturate the extraction solvent with guaiacol in the very first extraction step.

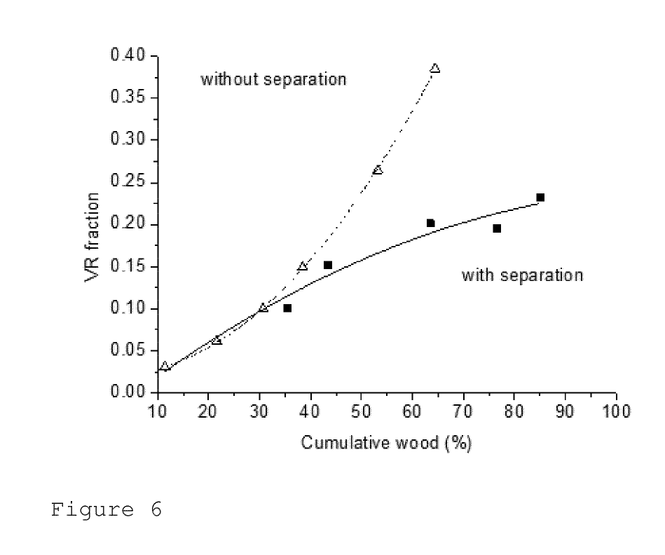

Having shown the principle of recovering the light bio-oil from the liquefaction, the extraction process was integrated into a liquefaction scheme to preferentially recycle the light bio-oil to minimize both the build-up of heavy components and the increase in the viscosity of the liquefaction medium. Hexadecane was used here as extractive solvent. As the number of liquefaction-separation cycles increased and the cumulative amount of wood processed (per gram of fresh solvent) increased, the VR fraction in the liquid reaction product increased only moderately (FIG. 6). This increase was much slower than reported previously upon recycle of the full liquefied product stream, without intermediate withdrawal of the heavy components (FIG. 6).

### Conclusion of Examples 1-4

For liquid-liquid extraction of bio-oil, solvents with either a very high or a very low polarity compared to guaiacol were found to be suitable solvents for separation of the light components from a liquefied product stream. In particular, water-methanol mixtures and hexadecane were found to be suitable extractive solvents which showed affinity towards light bio-oil at elevated temperature (>40° C.) but appeared to demix from the light bio-oil at lower temperature. This allowed the use of a temperature swing to recover the extractive solvent, which is expectedly more energy efficient than conventional distillation for solvent recovery. The potential of light bio-oil extraction was further demonstrated successfully through a series of liquefaction runs with intermediate solvent extraction and recycle.

### Example 5: Refinery Stream (LCO) Both as Liquefaction Solvent and as Extractive Solvent

In this example, the potential was investigated of using a refinery stream (LCO) as solvent in liquefaction and applying liquid-liquid extraction as separation system to recover the bio-oil from the refinery stream, to send the former for upgrading and recycle the latter to the liquefaction reactor.

For the liquefaction reaction, fine pine wood was used with a particle size of not more than 0.5 mm. Pine wood was milled and dried in an oven at 105° C. for 24 hours. Solvent for the liquefaction process (LCO—Light Cycle Oil) was obtained from Shell.

For the liquefaction reaction a batch set-up reactor was used with internal volume of 560 ml (see F. De Miguel Mercader in Pyrolysis oil upgrading for Co-processing in standard refinery units, Vol. Enschede, 2010, p. 176). For safety reasons, the reactor was placed in a high pressure bunkers. The operations during the reaction could be monitored and controlled from outside the chamber.

Refill liquefaction experiments were performed in the 560 ml reactor for 5 times. Liquefaction took place at 325° C. during 15 min. The liquid product was cooled to room temperature to allow for rejection of both light and heavy bio-oil from the LCO. The regenerated LCO could then be recycled to the autoclave for subsequent liquefaction experiments. The products yields were determined (See Table 4). Additionally, the amount of make-up solvent and the filtered liquid fractions after the reaction were determined. It was found that the VR fraction of the bio-oil in the refill experiments was essentially stable at around 0.4. The product quality data are shown in Table 5.

TABLE 4
Product distribution - refill with LCO

| Process stage | Gas (%) | Solids (%) | Oil (%) |
|---|---|---|---|
| initial round | 4 | 48 | 47 |
| 1^{st} refill | 5 | 40 | 55 |
| 2^{nd} refill | 5 | 40 | 55 |
| 3^{rd} refill | 5 | 49 | 46 |
| 4^{th} refill | 5 | 42 | 52 |

TABLE 5
Product quality

| | | | | ‘Bio-oil’ |
|---|---|---|---|---|
| Process | | | Viscosity | in LCO |
| stage | VR fraction | MCRT w % | (cP @ 50° C.) | (Mw >150 Da) |
| initial | 0.40 | 33 | — | 0.22 |
| round | | | | |
| 1^{st} refill | 0.42 | 29 | — | 0.27 |
| 2^{nd} refill | 0.41 | 25 | — | 0.29 |
| 3^{rd} refill | 0.40 | 29 | — | 0.31 |
| 4^{th} refill | 0.36 | 27 | 1328 | 0.32 |

According to tables 4 and 5, the liquefaction yields and quality of the liquefaction product (e.g. VR fraction and coking tendency (MCRT=Micro Carbon Residue test)) are fairly constant over several refills upon successive refill. The viscosity (measured at 50° C.) achieved after four refills amount to some 1300 cP, which is much lower than the viscosity of 3000-4000 cp (measured at 98 C) that was reported with recycle of the whole oil without heavy bio-oil removal (see WO2013/072383). Similarly, the quality of recycled LCO stream was fairly constant with only marginal increase in ‘bio-oil’ content to ˜0.3, defined as fraction with Mw above 150 Da.

This all shows that pine wood can be liquefied in LCO in recycle mode with easy separation of the LCO and the light and heavy bio-oils. The simultaneous separation of light and heavy bio-oil is meant here to prove the steady-state operation of the liquefaction. For commercial production of bio-oil, however, it will be advantageous to recover the light and heavy bio-oil separately using a two-stage cooling and liquid-liquid separation to allow separated upgrading of the bio-oils fractions.

### Example 6—Fractionation of Crude Oil

### Materials:

5 g of Basra medium crude oil with an API gravity of around 29.5 (crude oil coming from oil fields near Basra, Iraq, being one of the major Middle East crudes) and ˜16 g of solvent

The Hildebrand parameter for Basra crude was assumed to be around 17.5 [J/ml]^{0.5} by assuming that it consists of aliphatic and aromatic components which have Hildebrand of ˜16.5 [J/ml]^{0.5} and ˜18.5 [J/ml]^{0.5}, respectively.

Accordingly, the extraction was carried out using n-butanol as solvent because its has a Hildebrand (H) solubility parameter of 23.2 [J/ml]^{0.5}, i.e. about 5 (H) points higher than that of crude oil. Solvents with lower Hildebrand solubility parameters (e.g. Methanol with H=29.6 [J/ml]^{0.5}) indeed did not extract the light components while solvents with lower Hildebrand solubility parameters (e.g. 1-octanol with H=20.6 [J/ml]^{0.5}) dissolved too much of the heavy fraction.

The extraction experiments consisted of a few consecutive extractions at 50 or 60° C. using about 5 g of fresh crude oil (Basra) and about 16 g of solvent, followed by solvent regeneration by cooling at room temperature and allowing for L/L demixing using a centrifuge. The solvent consists of fresh n-butanol for the first run and recycled solvent for the subsequent runs.

The detailed experimental data can be found in Table 6. Accordingly, the 60° C. fractionation produced about 35% of raffinate with a VR fraction of about 0.6. The extract was clearly enriched in lighter product as indicated by a low vacuum residue fraction around 0.4. When carried out at 50° C., however, the fractionation produced slightly more raffinate (around 50%) with marginally lower VR fraction (0.56).

TABLE 6
Detailed Experimental data. Solvent: 1-Butanol.

| | | Basra | | | Solvent | VR fract. | VR fract. | VR fract. |
|---|---|---|---|---|---|---|---|---|
| | Solvent | crude | Extract | Raffinate | reg. | Extract | Raffinate | Solvent |
| | g | g | g | g | g | (Basra crude = 0.41) | | |
| T = 60° C. | | | | | | | | |
| Stage-1 | 16.1 | 5.1 | 2.3 | 1.4 | 17.1 | 0.50 | 0.65 | 0.17 |
| | | | (45%) | (27%) | | | | |
| Stage-2 | 16.3 | 5.1 | 3.2 | 1.9 | 16.1 | 0.42 | 0.61 | 0.19 |
| | | | (63%) | (37%) | | | | |
| Stage-3 | 15.5 | 5.0 | 4.3 | 1.7 | 14.5 | 0.39 | 0.60 | 0.18 |
| | | | (86%) | (34%) | | | | |
| T = 50° C. | | | | | | | | |
| Stage-1 | 16.2 | 5.0 | 1.0 | 2.8 | 17.2 | 0.46 | 0.62 | — |
| | | | (20%) | (56%) | | | | |
| Stage-2 | 17.2 | 5.1 | 2.3 | 2.7 | 17.1 | 0.40 | 0.56 | 0.18 |
| | | | (45%) | (53%) | | | | |

1. A process for liquid-liquid extraction of an oil-blend of non-uniform oligomeric and polymeric components, wherein “non-uniform” means that the components may have a varying size, shape and mass distribution, the process comprising a separation step wherein heavy components and light components in the oil-blend having similar chemical functionalities are separated to produce a heavy components fraction and a light components fraction, wherein the process comprises the steps (a) to (e), said method comprising:
(a) preselecting a desired molecular weight (Mw) boundary between heavy and light components;
(b) selecting an extractive solvent or an extractive mixture of solvents, which form essentially a single phase with the light components, such that at least 80% of the light components are dissolved, at elevated temperature, being the fractionation temperature, and in which the heavy components are essentially immiscible at the fractionation temperature, such that at most 10% of the heavy components are dissolved at said temperature in an amount of the extractive solvent/mixture of solvents in which the light components are fully dissolved at that temperature;
(c) mixing the oil-blend and the extractive solvent or extractive mixture of solvents selected in step (b) at elevated temperature, which is at least at or above said fractionation temperature, and wherein the extractive solvent/mixture of solvents to oil-blend ratio is from 1:2 to 100:1;
(d) allowing a phase split to form between the heavy components fraction and the light components/extractive solvent fraction at the fractionation temperature or at most 10° C. below the fractionation temperature;
(e) followed by separation of said fractions.

2. A process according to claim 1, wherein the extractive solvent/mixture of solvents in step (b) is selected such that it not only forms essentially a single phase with the light components at the fractionation temperature, but that it also demixes from the light components at a lower temperature, being the demixing temperature and wherein the separation step (e) is followed by cooling of the light components/extractive solvent fraction to the demixing temperature or lower and allowing demixing thereof, subsequently followed by a further separation step (f) to recover a light components stream and the extractive solvent/mixture of solvents.

3. A process according claim 1, wherein the oil-blend of non-uniform oligomeric and polymeric components is a bio-oil.

4. A process according to claim 3, wherein the extractive solvent or extractive mixture of solvents have a low polarity with log P>1, in particular up to log P=10.

5. A process according to claim 4, wherein the extractive solvent/mixture of solvents are selected from C6-C16 (cyclo)alkanes or mixtures thereof.

6. A process according to claim 5, wherein the extractive solvent is hexadecane.

7. A process according to claim 3, wherein the extractive solvent or extractive mixture of solvents have a high polarity with a Hildebrand solubility parameter from 37, in particular up to 41 [J/ml]^{0.5}.

8. A process according to claim 7, wherein the extractive mixtures of solvents are water-methanol mixtures.

9. A process according to claim 8, wherein the water-methanol mixtures have a volume ratio of 1:2 to 2:1, and preferably a volume ratio of 3:2.

10. A process according to claim 1, wherein the extractive solvent/mixture of solvents to bio-oil ratio used is from 1:2 to 10:1, preferably from 1:1 to 5:1.

11. A process according to claim 1, wherein the Hansen interaction radius Ra>16.

12. A process for liquefying a cellulosic material comprising:
a step (i) wherein the cellulosic material is contacted with a solvent or mixture of solvents to produce a liquefied product stream, and
a recycle step (ii) in which at least a part of the liquefied product stream is recycled to step (i),
wherein the process comprises
a process for liquid-liquid extraction according to claim 3 to extract the liquefied product stream, wherein at least a part of the light components fraction obtained in step (e) is recycled to step (i) as at least a part of the liquefied product stream that is recycled in step (ii).

13. A process according to claim 12, wherein the separation step (e) is followed by cooling of the light components fraction and demixing thereof, subsequently followed by a further separation step to recover a light components stream and the extractive solvent/mixture of solvents and wherein at least a part of the light components stream is recycled to step (i).

14. A process according to claim 13, wherein the separation step (e) is followed by cooling of the light components fraction and demixing thereof, subsequently followed by a further separation step to recover a light components stream and the extractive solvent/mixture of solvents and wherein the process comprises a step in which the extractive solvent/mixture of solvents is recycled to step (i) to be re-used as liquefaction solvent.

15. A process according to claim 14, wherein the solvent in step (i) and the extractive solvent/mixture of solvents is the same solvent and is Light Cycle Oil.
